(19) United States
(12) Patent Application Publication
(10) Pub. No.: US 2008/0206846 A1
(43) Pub. Date: Aug. 28, 2008

(54) Thermocycler

(75) Inventors: Henner Tasch, Hamburg (DE); Bernd Kruger, Tangstedt (DE)

Correspondence Address: RANKIN, HILL & CLARK LLP, 38210 Glenn Avenue, WILLOUGHBY, OH, 44094-7808, US

(73) Assignee: Eppendorf AG, Hamburg (DE)

(21) Appl. No.: 11/916,622

(22) PCT Filed: Mar. 4, 2006
(86) PCT No.: PCT/EP06/01995

§ 371 Date: Jan. 25, 2008

(30) Foreign Application Priority Data
Jun. 14, 2005 (DE) 10 2005 027 555.9

(51) Int. Cl. C12M 1/34

(52) U.S. Cl. 435/286.2

(57) ABSTRACT

A thermocycler is provided that includes a temperature-controlled receiving segment for reaction vials, a top which can be assembled to said base and which in the assembled state may be moved from an open position into an operational position above the receiving segment, an optics configured in the top, where the optics and/or said receiving segment are configured with play in the top respectively the base, the play allowing a relative displacement between the optics and the receiving segment sufficient to adjust the optics relative to the receiving segment, and at least 2 positioning elements are present at each base and each top to allow reproducible alignment between the optics and the receiving segment in a mutually adjusted position.

### BACKGROUND OF THE INVENTION

The present invention relates to a thermocycler. The term “thermocycler” in general denotes an apparatus for carrying out PCR (reactions). The abbreviation PCR is known to the expert as a method using polymerase allowing amplifying target nucleic acids contained in a test sample.

The present invention relates to thermocyclers allowing measuring in real time and over time the increase in the amplified products. Such thermocyclers comprise a base fitted with a temperature-controlled receiving segment receiving reaction vials suitable to carry out the PCR.

Such apparatus moreover is fitted with a top displaceable from an open position into an operational one above the receiving segment. The top contains an optics which in its operational position is opposite the receiving segment and acts as a transceiver radiating light into the reaction vials and receiving the light issuing from them.

As a rule fluorescent substances are contained in real-time PCR batches, the optics transmitting exciting light into the reaction vessels and detecting any emitted fluorescence.

In general several light paths are designed into the optics, each light path being allocated to a particular reaction vial seat in the receiving segment. To assure error-free measurement, the optics and the receiving segment must be accurately aligned to each other during PCR.

Most cases preclude factory adjustment. Illustratively, on account of weight, the top and the base are generally shipped separately. Also, the base is not necessarily restricted to real time procedures, but also may be used with other tops for conventional PCR.

Accordingly, in many applications, the top shall be mounted only subsequently on the base, namely after both units have left the factory.

The objective of the present invention is to create a thermocycler allowing in especially simple manner lasting/permanent adjustment between the optics and the receiving segment also when they are assembled subsequently.

### BRIEF SUMMARY OF THE INVENTION

In the thermocycler of the present invention, the optics and/or the receiving segment are seated with play in the top respectively the base. This play must be selected in a manner to allow adequate relative displacement between the optics and the receiving segment to adjust these relative to each other.

Moreover the base and the top each are fitted with at least 2 positioning elements, each positioning element at the base being associated with one positioning element at the top. At least one of these two mutually associated elements is adjustable in position relative to the receiving segment or the optics and once in its adjusted position can be affixed in place relative to the said receiving segment and/or optics.

One substantive effect of the present invention is that the receiving segment and/or the optics are mounted with play in the base respectively to the top, which is the sine qua non condition allowing adjustment for instance following or during top assembly. As elucidated farther below, the adjustment may be implemented by means of a gauge deposited on the receiving segment and aligning the optics when the top is lowered into its operational position.

The positioning elements are used to allow repeated arrangement of optics and receiving segment in the aligned position without readjustment during operation of the thermocycler.

During adjustment, the positioning elements of the thermocycler of the present invention are aligned relative to each other and affixed in their set position, that is, when they are received in displaceable manner. Following their affixation, all positioning elements are stationary in their positions relative to the receiving segment respectively the optics. For operation, it suffices to mutually align the positioning elements, as a result of which the optics and the receiving segment shall be automatically adjusted relative to one another.

In operation, the above discussed adjustment takes place in real time. In order not to design the base—which is not inevitably operated in real time—in unnecessarily complex manner, the elements required for adjustment are fitted merely into the top. In a preferred embodiment of the present invention, only the optics shall be received with play in the top, the receiving segment being seated in fixed manner in the base.

Conceivably there shall be a series of different, appropriate positioning elements. Illustratively such positioning elements may be merely detectable marks which, once the top is in the operational position, only need be mutually centered. Also illustratively, LED's might be used as the positioning elements while associated perforated templates are configured in the top. In that case a detector system might measure the light intensity passing through the said perforations and the adjusted position might be set for instance by displacing the optics until a maximum light intensity has been detected. Also magnetic or electric positioning elements might be used among many such approaches.

Preferably however such positioning elements shall be used that, when the top is moved into its operational position, will mechanically engage said top and thereby mutually align the receiving segment and the optics into their mutually adjusted position.

In this regard, an especially preferred embodiment of the present invention employs for instance guide pins acting as said guide elements illustratively configured at the base and engaging bushes configured at the top in geometrically locking manner.

As already mentioned above, the base may also be used for other than real time purposes; illustratively—instead of the optics being received in the top with play while the receiving segment is configured firmly affixed in the base—only the positioning elements fitted on the top area may be adjustable and affixable in their distance from the optics, whereas the positioning elements at the base cannot be adjusted, that is they are permanently affixed in place.

Furthermore and as already mentioned above, preferably a gauge is used during assembly when adjusting the optics and the receiving segment. This gauge illustratively is mounted in defined manner on the receiving segment and for instance is fitted upward pointing guide edges that shall engage associated mating walls at the lower optics end which they then shall commensurately displace into its adjusted position.

The present invention is elucidated below in relation two Figures.

### BRIEF DESCRIPTION OF THE DRAWINGS

FIG. 1 diagrammatically shows a sectional front view of the thermocycler of the present invention, the optics being shown in section along line I-I of FIG. 2.

FIG. 2 is a top view of the optics of the thermocycler of the present invention.

### DETAILED DESCRIPTION OF THE INVENTION

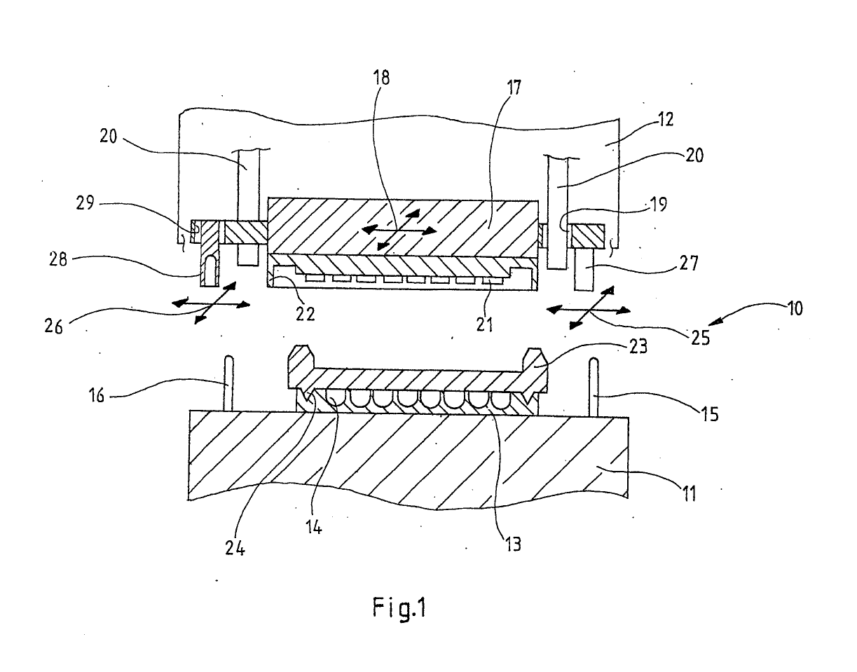

FIG. 1 shows a thermocycler 10 comprising a base 11 and a top 12. The base 11 is fitted with a temperature-controlled receiving segment 13 fitted with reaction vial wells 14.

Positioning elements in the form of pins 15 and 16 are mounted on the base 11. The positioning elements 15 and 16 are stationary and are spaced away from the receiving segment 13 which also is stationary.

An optics 17 is supported with play in the direction of the arrow 18 in the top 12 by means of boreholes 19 receiving retention bars 20. The diameter of the boreholes 19 slightly exceeds that of the bars, subtending thereby said play.

Measuring apertures 21 are configured at the lower end of the optics 17. Also, said lower end is fitted with a frame 22. The frame 22 is shaped in a manner that it can engage in adjusting manner a gauge 23 which, in the embodiment shown, can be deposited in predetermined manner, by means of studs 24, on the receiving segment 13.

As the top 12 is lowered, the frame 22 will engage the gauge 23 and thereby will displace the optics 17, in this case to the right, as a result of which the optics 17, that is its measuring apertures 21, shall then be adjusted for proper alignment with the reaction vial wells 14.

Furthermore positioning elements designed as bushes 27 and 28 displaceable in grooves 29, 30 in the direction of the arrows 25 and 26 are configured in the top.

During the alignment of the optics 17 with the receiving segment 13, the bushes 27 and 28 are moved to geometrically interlock with the pins 15 and 16; this feature is easily assured by the displaceability of said bushes.

As soon as the optics unit 17 is adjusted by the gauge 23 relative to the receiving segment 13 and once the pins 15 and 16 have trapped the bushes 27 and 28, the latter can be affixed by omitted elements such as screws etc. into their particular positions. Thereupon the bushes 27 and 28 no longer are displaceable in the directions of the arrows 25 and 26. Instead they are stationary relative to the optics 17 and can only be moved jointly with said optics in the direction of the arrow 18.

Accordingly the adjusted position of the optics 17 determined by the gauge 23 is reproducible at every descent in that the pins 15 and 16 engage the bushes 27 and 28.

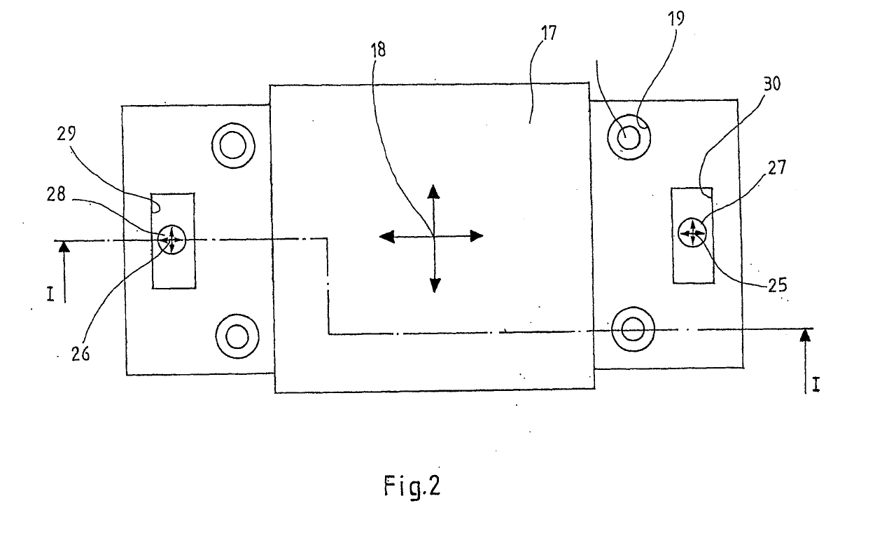

Referring to FIG. 2, FIG. 2 shows a top view of the optics of FIG. 1. The optics 17 are supported by means of four boreholes 19 at retaining bars 20 with play in the direction of the arrow 18. The bushes 27 and 28 are adjustable in grooves 29, in the directions of the arrows 26 and 25 and can be affixed in a set position by an omitted element.

1: A thermocycler comprising:
a base fitted with a temperature-controlled receiving segment of reaction vials, the base having a top which can be mounted on the base, which in its assembled state can be displaced from an open position wherein the receiving segment is accessible for purposes inserting and removing the reaction vials, into an operational position above the receiving segment;
an optics configured in the top, wherein the optics when in its operational position is radiating light into the reaction vials and/or detecting light issuing from the reaction vessels,
wherein the optics and/or the receiving segment are received with play in the top respectively the base, the play allowing relative movement between the optics and the receiving segment sufficient for adjustment of the optics and the receiving segment and in that at least 2 positioning elements are present both in the base and in the top,
wherein each positioning element at the top being associated with a positioning element at the base and in that at least one of these two mutually associated positioning elements is adjustable in position relative to the receiving segment or to the optics and is affixable in a set position to be stationary relative to the receiving segment and/or the optics in a manner that adjusting the optics relative to the receiving segment allows mutually aligning the associated positioning elements into permanent affixation.

2: The thermocycler as claimed in claim 1, wherein only the optics is received with play in the top.

3: The thermocycler as claimed in claim 1, wherein when the top is moved into its operational position, the positioning elements automatically align the receiving segment and the optics into their adjusted position.

4: The thermocycler as claimed in claim 3, wherein the mutually associated positioning elements are bushes and pins that engage each other in mutually interlocking manner.

5: The thermocycler as claimed in claim 3, wherein only the positioning elements configured at the top can be adjusted in position.

6: The thermocycler as claimed in claim 1, wherein the optics comprises elements allowing adjusting it relative to the receiving segment by means of a gauge mounted on said segment.
